(19) United States
(12) Patent Application Publication
(10) Pub. No.: US 2003/0027175 A1
(43) Pub. Date: Feb. 6, 2003

(54) Dynamic whole genome screening methodology and systems

(76) Inventors: Gregory Stephanopoulos, Winchester, MA (US); Ryan T. Gill, Boston, MA (US); Stefan Wildt, Cambridge, MA (US)

Correspondence Address: ROPES & GRAY, ONE INTERNATIONAL PLACE, BOSTON, MA, 02110-2624, US

(21) Appl. No.: 10/076,106

(22) Filed: Feb. 13, 2002

Related U.S. Application Data
(60) Provisional application No. 60/268,638, filed on Feb. 13, 2001.

(51) Int. Cl. C12Q 1/68; C12P 19/34

(52) U.S. Cl. 435/6; 435/91.1

(57) ABSTRACT

The present invention relates to a methodology for discovering genes responsible for a particular phenotype. The subject method can be used with a variety of cell types, including eukaryotic and prokaryotic cells, and for determining the role of genes and gene programs in the various growth states of those cells and responses to drugs or other environmental cues.

### RELATED APPLICATIONS

This application claims priority to U.S. Provisional application U.S. Ser. No. 60/268,638 filed Feb. 13, 2001, the specification of which is incorporated by reference herein.

### BACKGROUND OF THE INVENTION

Identification, sequencing and characterization of genes is a major goal of modem scientific research. By identifying genes, determining their sequences and characterizing their biological function, it is possible to employ recombinant technology to produce large quantities of valuable gene products, e.g. proteins and peptides. Additionally, knowledge of gene sequences can provide a key to diagnosis, prognosis and treatment in a variety of disease states in plants and animals which are characterized by inappropriate expression and/or repression of selected genes or by the influence of external factors, e.g., carcinogens or teratogens, on gene function.

Genes which are essential for the response of an organism to its environment, such as growth cues or drugs, however, have been difficult to identify in such a manner as to be easily recovered for future analysis. The most common methodology currently employed to identify essential genes is a multi-step process involving the generation of a conditionally lethal mutant library followed by the screening of duplicate members under the appropriate permissive and non-permissive conditions. Candidate mutants are then transformed with a second, genomic library and the desired genes isolated by complementation of the mutant phenotype. The complementing plasmid is recovered, subcloned, and then retested. However, this procedure comprises multiple subcloning steps to identify and recover the desired genes thus making it both labor intensive and time consuming.

To further illustrate, knowledge of genes or gene products essential to the growth of an organism can provide a key to the development of treatments of infectious pathogens.

### BRIEF SUMMARY OF THE INVENTION

The present invention relates to a methodology for discovering genes responsible for a particular phenotype. For ease of reference we have termed this technique MADSA: Micro-Array Dynamic Screening Approach. The subject method can be used with a variety of cell types, including eukaryotic and prokaryotic cells, and for determining the role of genes and gene programs in the various growth states of those cells and responses to drugs or other environmental cues.

One aspect of the invention provides a method for identifying one or more genetic elements which selectively confer a target phenotype on a target cell. In one embodiment, host cells are transfected with a library of expression vectors comprising a variegated population of coding sequences for genetic elements. The host cells are subjected to selective growth conditions, e.g., under which genetic elements are also expressed. A sub-population of those host cells having the target phenotype during selective growth conditions are isolated and/or amplified from the culture. Genetic elements from the isolated/amplified sub-population of host cells are contacted with one or more oligonucleotide arrays, and the individual coding sequences of the isolated genetic elements determined based on hybridization to said arrays.

In a certain preferred embodiments, the subject begins with obtaining or generating a library of expression vectors for a variegated population of genetic elements derived from genomic fragments. The expression vector library is transfected into host cells, and the host cells subjected to selective growth conditions, e.g., under which genetic elements are also expressed. A sub-population of those host cells having the target phenotype during selective growth conditions are isolated and/or amplified from the culture. Genetic elements from the isolated/amplified sub-population of host cells are labeled, as are genomic fragments (different lable from sub-population), the two different labeled population mixed, and the admixture contact with one or more oligonucleotide arrays under conditions in which genetic elements and genomic fragments in the mixture can selective hybridize with complementary sequence in the array(s). From the pattern of hybridization, the coding sequences which confer the target phenotype can be determined.

The genetic elements can be genomic fragments, cDNA sequences, antisense sequences, and transcriptional regulatory elements.

The host cell can be prokaryotic cell or eukaryotic cell.

The expression vector can be an episomal vector or an intregrative vector.

Exemplary target phenotypes include changes in the level of expression of a reporter gene, such as a fluorescent marker, or gene product which confers resistance to a particular culture condition, such as antibiotic resistance. The target target phenotype can be manifest by a change in the level of expression of a marker protein, such as a cell surface antigen or other antigenic determinant. The target phenotype can also be a gross change in structure of the cell (morphology) or hemostasis of the cell, e.g., ability to growth generally or under selective conditions.

In certain preferred embodiments, the sub-population of host cells having the target phenotype is isolated by flow cytometry, such as when the target phenotype includes expression of a FACS marker or antigenic determinant which can be fluorescently labeled on the whole cell.

Another aspect of the present invention provides a method of conducting a drug discovery business. For instance, the business method includes a program for identifying, e.g., by the subject MADSA system, one or more target genes which confer a target phenotype. Those target genes individually or in various combinations can be used as drug screening targets, e.g., to develop agents which inhibit or potentiate (as the case may be) the activity of the target genes' products. Agents are identified by their ability to inhibit or potentiate expression of the target gene or the activity of an expression product of the target gene in order to inhibit or promote, as appropriate, the target phenotype in a target cell or tissue. The method further includes the steps of conducting therapeutic profiling of agents identified in the steps above, or further analogs thereof, for efficacy and toxicity in animals. Then the method formulates pharmaceutical preparations including one or more agents identified as having an acceptable therapeutic profile.

In certain embodiments, the subject business method may include an additional step of establishing a distribution system for distributing the pharmaceutical preparation for sale, and may optionally include establishing a sales group for marketing the pharmaceutical preparation.

In another embodiment, the business method includes a program for identifying, e.g., by the subject MADSA system, one or more target genes which confer a target phenotype. As an optional step, the method can include conducting therapeutic profiling of the loss-of-function or gain-of-function phenotypes of said target gene with respect to potential efficacy and toxicity in animals. The rights for further drug development of inhibitors of said target gene(s) are licensed to one or more third parties.

### BRIEF DESCRIPTION OF THE DRAWINGS

FIG. 1: Micro-array Dynamic Screening Approach (MADSA).

FIG. 2: Growth of DH5a WT and DH5a [pTAGL] in Luria Broth+Ampicillin (LBA) supplemented with Pine-Sol (0.0-1% v/v). At <0.1% (v/v) Pine-Sol growth was not effected by the presence of Pine-Sol. Between 0.1-1%, the lagtime was increased and the final cell density was decreased. At >1% Pine-Sol, no cell growth was observed for greater then 35 hours. The addition of the genomic library improved growth versus the wild-type at Pine-Sol concentrations between 0.1-0.4% (v/v) but did not grow substantially different than the wild-type when Pine-Sol was at low levels (<0.1%) or not present in the media.

FIG. 3: DNA micro-array images taken from plasmid samples from mid-exponential and early stationary phase of 0.4% Pine-Sol growth and from early stationary phase of 0.0% Pine-Sol growth (control). The green signals correspond to plasmid samples labeled with Cy3-dUTP and the red signals correspond to the control genomic DNA labeled with Cy5-dUTP and added in equal proportions to each array. Blocks 1, 6, and 8 are representative blocks from the full 8 block array. The patterns of bright green spots in the 0.4% arrays reveal the presence of a selected sub-population of clones not apparent in the 0.0% samples. These spots were reproducibly observed at two additional time points from early exponential phase and early stationary phase of the 0.4% cultures but were not observed in any of the 0.0% cultures.

FIG. 4: Quantified intensity values for the early stationary phase arrays displayed in FIG. 3. Cy3 intensity values (green color of FIG. 3) are located along the y-axis and the Cy5 intensity values (red color of FIG. 3) are represented along the x-axis. The scales were set to be equal in both Figures to emphasize the similar distribution of Cy5 signals (control genomic DNA) and the dramatically different distribution of Cy3 intensities. The genes with the largest Cy3 intensity correspond to genes that were selected for in the presence of Pine-Sol but were clearly not selected for in an identical growth conditions without the presence of Pine-Sol. These genes are Pine-Sol resistance genes.

### DESCRIPTION OF PREFERRED EMBODIMENTS

I. Overview

Current functional genomic technologies identify genes differentially regulated as a result of a specific physiology or phenotype (see references 1, 2). It is often desired, however, to determine genes whose overexpression results in a specific physiology or phenotype. The present invention relates to a methodology that provides this information by screening entire genome libraries in a heterogeneous culture without the need for sequencing.

Genome sequencing projects are progressing at tremendous pace. To keep up with the generation of sequence information, new techniques for assigning function to these genes must be developed. Furthermore, new data that enable an integrative genomics approach are required. The technique of the present invention is complementary, but substantially different, from traditional expression profiling (see references 1, 2, 23, 24). In expression profiling, genes up or down regulated as a result of a specific physiology are monitored. In the described approach, genes whose overexpression result in a specific physiological phenotype are identified.

II. Definitions

For convenience, certain terms employed in the specification, examples, and appended claims are collected here.

As used herein, the term “vector” refers to a nucleic acid molecule capable of transporting another nucleic acid to that it has been linked. One type of vector is a genomic integrated vector, or “integrated vector”, which can become integrated into the chromsomal DNA of the host cell. Another type of vector is an episomal vector, i.e., a nucleic acid capable of extra-chromosomal replication. Vectors capable of directing the expression of genes to that they are operatively linked are referred to herein as “expression vectors”. In the present specification, “plasmid” and “vector” are used interchangeably unless otherwise clear from the context.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

The term “genetic element” is meant to include genes, gene products (such as RNA molecules, and polypeptides), cis-acting regulatory elements (such as promoter elementsand enhancer elements). The method allows differences in the patterns of expression of any of these molecule types to be evaluated, and put into a biological context in the light of the cellular process that is being studied. The method also allows differences in the constituent genetic elements to be investigated, for example, to identify mutations and polymorphisms that affect the biological response to a particular cellular process.

As used herein, the term “gene” or “recombinant gene” refers to a nucleic acid comprising an open reading frame encoding a polypeptide of the present invention, including both exon and (optionally) intron sequences.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

However, as described below, the generic term “coding sequence” may refer to, as the context permits, sequences that are transcribed to produce RNA that is itself directly active (as a potential genetic element), as opposed to a polypeptide translated therefrom.

Likewise, “encodes”, unless evident from its context, will be meant to include DNA sequences that encode a polypeptide, as the term is typically used, as well as DNA sequences that are transcribed into inhibitory antisense molecules.

An “genetic element library” is a library of coding sequences for potential genetic elements.

The term “loss-of-function”, as it refers to genetic elements, refers to those elements that inhibit expression of a gene, or render the gene product thereof to have substantially reduced activity, or preferably no activity relative to one or more functions of the corresponding wild-type gene product.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence. On the other hand, “expression” of an antisense sequence or ribozyme will be understood to refer to the transcription of the recombinant gene sequence as it is the RNA product that is directly active.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “heterologous nucleic acid” in the present context means that the nucleic acid is not present in its natural context i.e. the host cell has been modified so as to contain the nucleic acid which would otherwise not be present in the form in which it is introduced.

As used herein, the terms “transduction” and “transfection” are art recognized and mean the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, where anti-sense expression occurs from the transferred gene, the expression of a naturally-occurring form of a protein is disrupted.

“Transient transfection” refers to cases where exogenous DNA does not integrate into the genome of a transfected cell, e.g., where episomal DNA is transcribed into mRNA and translated into protein.

A cell has been “stably transfected” with a nucleic acid construct when the nucleic acid construct is capable of being inherited by daughter cells.

An “oligonucleotide” includes a nucleic acid polymer composed of two or more nucleotides or nucleotide analogs. An oligonucleotide can be derived from natural sources but is often synthesized chemically. It is of any size.

An “oligonucleotide array” includes a spatially defined pattern of oligonucleotide probes on a solid support. A “preselected array of oligonucleotides” is an array of spatially defined oligonucleotides on a solid support.

A “solid support” includes a fixed organizational support matrix, such as silica, polymeric materials, or glass.

The term “label” refers to a composition detectable by, for example, spectroscopic, photochemical, biochemical, immunochemical, or chemical means.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences can be directly or indirectly regulated by the target receptor protein. Exemplary transcriptional control sequences are promoter sequences. A reporter gene is meant to include a promoter-reporter gene construct that is heterologously expressed in a cell.

As used herein, “growth”, “proliferating” and “proliferation” refer to cells undergoing mitosis.

The “growth state” of a cell refers to the rate of proliferation of the cell and the state of differentiation of the cell.

III. Illustrative Embodiments

In one illustrative embodiment, the subject method can be used to increase antibiotic resistance or susceptibility in pathogens. By “pathogen” it is meant any organism which is capable of infecting an animal or plant and replicating its nucleic acid sequences in the cells or tissue of the animal or plant. Such a pathogen is generally associated with a disease condition in the infected animal or plant. Such pathogens may include, but are not limited to, viruses, which replicate intra- or extracellularly, or other organisms such as bacteria, fungi or parasites, which generally infect tissues or the blood. Certain pathogens are known to exist in sequential and distinguishable stages of development, e.g., latent stages, infective stages, and stages which cause symptomatic diseases. In these different states, the pathogen is anticipated to rely upon different genes as essential for survival. Preferred pathogens useful in the methods of the invention include, for example, Streptococcus, Streptococcus pneumoniae, Staphylococcus, Staphylococcus aureus, Enterococcus, Enterococcus faecalis, Pseudomonas, Pseudomonas aeruginosa, Escherichia, and Escherichia coli.

While the appended examples demonstrate the strength of the present technique in analyzing microbial resistance and susceptibility within a single organism, it is specifically contemplated that there are many other applications of such a screening methodology. For example, it may be possible to screen libraries of pathogenic bacteria within a non-pathogenic host for genes displaying the properties of interest. In addition, it is often desirable to identify which genes in a given environment, when overexpressed rather than deleted (see references 13), result in a specific phenotype.

For instance, mammalian cell libraries can be screened to identify those genes which are able to help their respective hosts to survive the presence of a cell-death-inducing factor. Furthermore, for example, it might also be of interest to screen for genes, which make cancer cells more susceptible to certain agents. Finding of those genes might help to further elucidate the mechanisms of antibiotic-resistance, antibiotic-susceptibility, cancer and programmed cell-death and lead to the identification of drugs and/or drug-targets. Likewise, screens for overexpression strains that provide higher productivity due to better product formation, stress-resistance, increased or altered substrate utilization, or decreased product associated toxicity are envisioned. Moreover, most screening techniques only identify surviving clones (see references 12) even though the identity of those clones made more susceptible to the treatment are also of great interest. In all of these and many other cases, the approach described herein is applicable. The integration of this technique with many of the other functional genomic, proteomic, and bioinformatic approaches provides a powerful portfolio for the systematic evaluation of genome function and its relation to cell physiology.

A variety of other features and advantages to the subject method are briefly described to include:

1. Use of MADSA to improved productivity

a. Screen coexpression libraries with recombinant proteins

protein could be toxic if not folded or produced properly and non-toxic when folded and produced correctly

protein could be fused to fluorescent marker protein and technique combined with flow cytometry or similar selection technique in repeated rounds of MADSA/Flow Cytometry

b. Screen for overexpression libraries that improve Asparaginase production

For example, grow E. coli [pTAGL] strain that makes asparaginase in the presence of 1-asparaginine with very low or no glucose in the medium. As 1-asparaginine is converted to 1-aspartate cells can use 1-aspartate as carbon source, those cells which are able to do this conversion more efficiently should out compete rivals. Control is identical cells grown only in the presence of 1-aspartate. Asparaginase is an anticancer agent.

c. Screen against any toxic products or by-products (not just proteins)

d. Screen for product quality (chirality—growth retarding enantiomers)

e. Screen for enhancing subtrate utilization

2. Antibiotic Resistance and Susceptibility

a. As described below

b. Screen of pathogenic bacteria genomes in non-pathogenic host

3. Cancer Screens

a. Oncogene Screen: Create library in organism that can support plasmid (such as viral vectors) and undergoes apoptosis. Grow in the presence and absence of apoptosis inducing agent and identify genes which prevent apoptosis (oncogenes) or encourage apoptosis.

b. Chemotherapy Screen: Grow tumor cell line or potentially normal cells transformed with overexpression library from tumor cell genome. Add various chemotherapies and monitor cells which are more resistant to chemotherapies (oncogenes) or more susceptible (drug “enhancing” targets).

4. Plasmid Stability

a. Screen for genes which enhance the stability of plasmids in relevant bioprocesses

b. Screen for origins of replication which enhance plasmid stability (targeted towards bugs which do not normally like plasmids—synechocystis)

5. Bioprocess Applications

a. Screen for ultra-high cell density plasmids: Screen library for inserts that allow cells to grow to higher cell densities—could be non-host library (i.e. Khoslas work or putting hemoglobin gene into E. coli to obtain high densities better Oxygen uptake)

b. Screen for difficult condtion plasmids: Screen in oxygen limited, pH altered, temperature changed, differential osmolarity; “extreme or slightly extreme environments” for enriched plasmids. Again could be traditional host with non-traditional library to look for plasmids that expand the growth abilities.

6. Environmental Applications

a. Screen for degradation of toxic-compounds: again traditional host containing non-traditional host library but does not have to be.

b. Screen for degradation of compounds in extreme environments—radioactive environments such as those at Dept. of Energies nuclear waste sites.

7. Identification of the functional mechanism of action of toxic compounds. Using this screen, the enriched genes provide evidence about the “functions” that allow resistance to the toxic compound and hence the toxic mechanism—i.e. interferes with amino acid metabolism. A more functional mechanism in that rather than revealing that it binds with tRNA, it would reveal that cells capable of over-producing this class of amino-acids are enriched.

8. Identification of the chemical class of unknown toxic compounds/ mixtures. Given the results regarding Pine-Sol and the amino-acid pathways affected. We could get an idea that the active component was likely to be a 5-6 ring hydrocarbon. Could develop a database of enriched plasmids as a function of different chemical structures, then map unknown structures by the results of identical screens.

9. Network Reconstruction. We know from our results that 19 different genes provided the cell with the identical “functional” phenotype i.e. resistance to Pine-Sol. Those genes could be grouped together within a genetic network. The idea being to reconstruct regulatory networks and map unknown genes based on the phenotype resulting from their overexpression.

In preferred embodiments, to identify the isolated genetic elements, the present method utilizes immobilized DNA or oligonucleotide libraries, e.g., of known sequence, in an organized array. The GeneChip™ system (Affymetrix, Santa Clara, Calif.) is particularly suitable for identifying sequences in the sub-population; however, it will be apparent to those of skill in the art that any similar systems or other effectively equivalent detection methods can also be used. For instance, oligonucleotides can be bound to a solid support by a variety of processes, including lithography. These nucleic acid probes comprise a nucleotide sequence at least about 12 nucleotides in length, preferably at least about 15 nucleotides, more preferably at least about 25 nucleotides, and most preferably at least about 40 nucleotides, and up to all or nearly all of a sequence which is complementary to a portion of the coding sequence of one or more genes or other genomic elements of interest.

In one embodiment, the nucleic acid probes are spotted onto a substrate in a two-dimensional matrix or array. Samples of nucleic acids can be labeled and then hybridized to the probes. Double-stranded nucleic acids, comprising the labeled sample nucleic acids bound to probe nucleic acids, can be detected once the unbound portion of the sample is washed away.

The probe nucleic acids can be spotted on substrates including glass, nitrocellulose,etc. The probes can be bound to the substrate by either covalent bonds or by non-specific interactions, such as hydrophobic interactions. The sample nucleic acids can be labeled using radioactive labels, fluorophores, chromophores, etc.

Techniques for constructing arrays and methods of using these arrays are described in EP No. 0 799 897; PCT No. WO 97/29212; PCT No. WO 97/27317-EP No. 0 785 280;PCT No. WO 97/02357; U.S. Pat. Nos. 5,593,839; 5,578,832; EP No. 0 728520; U.S. Pat. No. 5,599,695; EP No. 0 721 016; U.S. Pat. No. 5,556,752; PCT No. WO95/22058; and U.S. Pat. No. 5,631,734.

The genetic element libraries can be generated by any of a number of techniques, including from genomic DNA fragments or from cDNA libraries. The libraries can be generated from the host cell, or other cells of interest.

In certain embodiments, the initial genetic element library can be a subtractive cDNA library. Many strategies have been used to create subtractive libraries, and can be readily adapted for use in the present method. One approach is based on the use of directionally cloned cDNA libraries as starting material (Palazzolo and Meyerowitz, (1987) Gene 52:197; Palazzolo et al. (1989) Neuron 3:527; Palazzolo et al. (1990) Gene 88:25). In this approach, cDNAs prepared from a first source tissue or cell line are directionally inserted immediately downstream of a bacteriophage T7 promoter in the vector. Total library DNA is prepared and transcribed in vitro with T7 RNA polymerase to produce large amounts of RNA that correspond to the original mRNA from the first source tissue. Sequences present in both the source tissue and another tissue or cells, such as normal tissue, are subtracted as follows. The in vitro transcribed RNA prepared from the first source is allowed to hybridize with cDNA prepared from either native mRNA or library RNA from the second source tissue. The complementarity of the cDNA to the RNA makes it possible to remove common sequences as they anneal to each other, allowing the subsequent isolation of unhybridized, presumably tissue-specific, cDNA. This approach is only possible using directional cDNA libraries, since any cDNA sequence in a non-directional library is as likely to be in the “sense” orientation as the “antisense” direction (sense and antisense are complementary to each other). A cDNA sequence unique to a tissue would be completely removed during the hybridization procedure if both sense and antisense copies were present.

Where cDNA libraries are used, it may also be desirable to utilizes normalized libraries, e.g., to reduce the percentage of otherwise abundant messages. U.S. Pat. 5,702,898 describes a method to normalize a cDNA library constructed in a vector capable of being converted to single-stranded circles and capable of producing complementary nucleic acid molecules to the single-stranded circles comprising: (a) converting the cDNA library in single-stranded circles; (b) generating complementary nucleic acid molecules to the single-stranded circles; (c) hybridizing the single-stranded circles converted in step (a) with complementary nucleic acid molecules of step (b) to produce partial duplexes to an appropriate Cot; (e) separating the unhybridized single-stranded circles from the hybridized single-stranded circles, thereby generating a normalized cDNA library.

In certain embodiments, it may be desirable to use a direction library, e.g., a directional cDNA library. In one directional cloning strategy, which can be used to generate an initial genetic element library, a DNA sequence encoding a specific restriction endonuclease recognition site (usually 6-10 bases) is provided at the 5′ end of an oligo(dT) primer. This relatively short recognition sequence does not affect the annealing of the 12-20 base oligo(dT) primer to the mRNA, so the cDNA second strand synthesized from the first strand template includes the new recognition site added to the original 3′ end of the coding sequence. After second strand cDNA synthesis, a blunt ended linker molecule containing a second restriction site (or a partially double stranded linker adapter containing a protruding end compatible with a second restriction site) is ligated to both ends of the cDNA. The site encoded by the linker is now on both ends of the cDNA molecule, but only the 3′ end of the cDNA has the site introduced by the modified primer. Following the linker ligation step, the product is digested with both restriction enzymes (or, if a partially double stranded linker adapter was ligated onto the cDNA, with only the enzyme that recognizes the modified primer sequence). A population of cDNA molecules results which all have one defined sequence on their 5′ end and a different defined sequence on their 3′ end.

A related directional cloning strategy developed by Meissner et al. (1987) PNAS 84:4171), requires no sequence-specific modified primer. Meissner et al. describe a double stranded palindromic Bam-HI/HindIII directional linker having the sequence d(GCTTGGATCCAAGC), that is ligated to a population of oligo(dT)-primed cDNAs, followed by digestion of the ligation products with BamHI and HindIII. This palindromic linker, when annealed to double stranded form, includes an internal BamHI site (GGATCC) flanked by 4 of the 6 bases that define a HindIII site (AAGCTT). The missing bases needed to complete a HindIII site are d(AA) on the 5′ end or d(TT) on the 3′ end. Regardless of the sequence to which this directional linker ligates, the internal BamHI site will be present. However, HindIII can only cut the linker if it ligates next to an d(AA):d(TT) dinucleotide base pair. In an oligo(dT)-primed strategy, a HindIII site is always generated at the 3′ end of the cDNA after ligation to this directional linker. For cDNAs having the sequence d(TT) at their 5′ ends (statistically 1 in 16 molecules), linker addition will also yield a HindIII site at the 5′ end. However, because the 5′ ends of cDNA are heterogeneous due to the lack of processivity of reverse transcriptases, cDNA products from every gene segment will be represented in the library.

In other embodiments, the genetic element library is generated from genomic DNA fragments. Preferably, the inserts in the library will range from about 100 bp to about 700 bp and more preferably, from about 200 bp to about 500 bp in size. Such genetic element libraries, in addition to encoding polypeptide and antisense molecules that may be functional genetic elements in the test method, may also “encode” decoy molecules, e.g., nucleic acid sequences which correspond to regulatory elements of a gene and which can inhibit expression of the gene by sequestering, e.g., transcriptional factors, and thereby competing for the necessary components to express the endogenous gene.

IV. Examples

We have screened a plasmid based genomic library for those genes which provide E. coli with increased resistance or susceptibility to the anti-microbial agent Pine-Sol using a DNA micro-array of 1160 E. coli genes. The results revealed 19 gene containing plasmids (resistance genes) that were enriched in the selective conditions. These same genes were not enriched in the antibiotic free cultures. In addition, 27 genes (susceptibility genes) were identified from transformants that grew poorly in the selective media when compared to the antibiotic free media.

It is often desirable that genes conferring a particular trait upon a cell be identified. For example, pathogenic organisms that develop resistance against antibiotics are possibly doing so because of alterations in one or more of their genes or regulatory regions (see reference 3). In other cases, industrial microorganisms overproduce product because of optimal activation of one or more critical genes or co-expression of foreign genes (see reference 4). Also, changes in the way some genes are expressed may result in the onset of disease or a favorable response to a therapeutic agent. In all of the above and many other situations the identification of those genes that are most critical for the observed cellular behavior is most important for drug screening, therapy development, or bio-process optimization.

In this application we demonstrate a technique that discovers those genes most responsible for the phenotype of increased antibiotic resistance or susceptibility (for ease of reference we have termed this technique MADSA: Micro-Array Dynamic Screening Approach). Antibiotic resistant strains of Staphylococcus aureus, Mycobacterium tuberculosis, and Streptococcus pneumonia among others have all been identified (see reference 5). These microbes are among the leading causes of bacterially based human disease and mortality (see reference 6). Each of these bacteria has recently been fully sequenced and studies of the function of these genomes will be the next step in the discovery of new drug targets and the development of novel drugs directed towards such strains (see reference 7). With an estimated worldwide market of close to $25 billion/yr. for antibiotics, the development of new anti-microbial agents is expected to play an increasingly important role in the pharmaceutical and biotechnology industries (see reference 5).

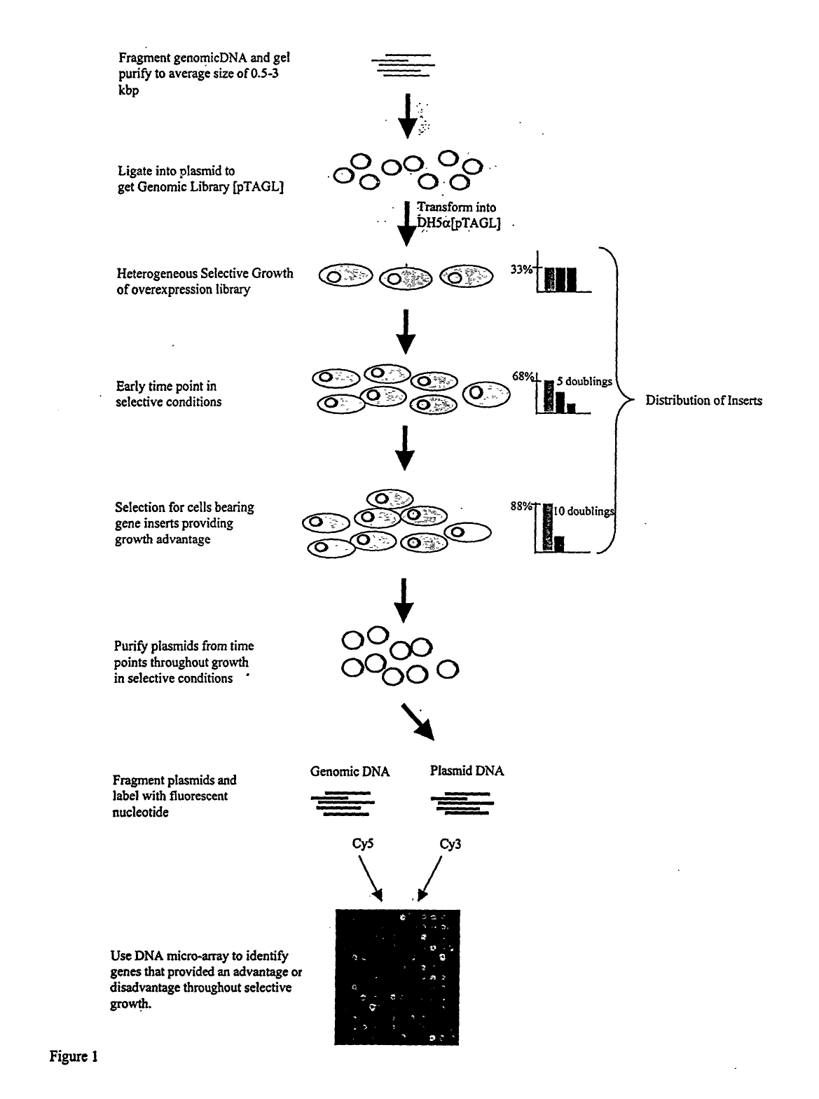

While development of new antibiotics of traditional classes is still of importance, current efforts are shifting towards the discovery of novel drug targets and new classes of antibiotics altogether. Here we report a new technique that provides these drug targets by screening a whole genome overexpression library on a DNA micro-array (see FIG. 1). Specifically, E. coli MG1655 genomic DNA was fragmented, size selected, repaired, and ligated into TOPO-TA cloning vector (see reference 8). E. coli DH5α were transformed with the ligation reaction and approximately 12,000 successful transformants were harvested and their plasmids purified (see reference 9). This library was used as the starting pool for all subsequent transformations. To perform the dynamic screen, chemically competent DH5α were transformed with the library and grown immediately in LB+Ampcillin (LBA) until an OD₆₀₀ of 0.1-0.2 (c.a. 5 hours) at which point they were inoculated (at 1% (v/v)) into selective (0.1-2.5% (v/v) Pine-Sol) and non-selective LBA. Assuming exponential growth kinetics, cells bearing plasmids that provide a growth advantage will become the predominant members of the culture. In fact, cells growing at only a 30% increased growth rate will be 3-fold enriched after 5 doublings and 8-fold enriched after 10 doublings (accounting for almost 90% of the culture assuming an evenly distributed starting culture). As a result, cells bearing plasmids that do not enhance growth or that retard growth will be rapidly lost from the culture. To probe changes in the plasmid population, samples were obtained throughout exponential and stationary phase growth, the plasmids were purified, fragmented by sonication, and labeled with Cy3-dUTP (see reference 10). In parallel, the original fragmented MG1655 genomic DNA was labeled with Cy5-dUTP to be used as a control. These two labeled pools of extra-chromosomal and genomic DNA were mixed, purified from unincorporated nucleotides, and hybridized to a E. coli DNA micro-array containing 1160 E. coli MG1655 genes (see reference 11). All resultant micro-array images contained the identical genomic DNA in the Cy5 channel thereby enabling a gene-by-gene internal control for each array. By monitoring changes in the plasmid population throughout growth in selective and non-selective conditions we identified those genes which provided a growth advantage or disadvantage and, hence, antibiotic resistance and susceptibility genes.

This technique differs from previous screening and micro-array techniques in a number meaningful ways. First, the majority of screening techniques rely upon timeconsuming sequencing of clones which survive a particular condition. In this technique, the sequencing portion of the screen is performed using the DNA micro-array. While this does save considerable time and expense, the real advantage is the ability to identify clones which do not survive the condition of interest (see reference 12). In the search for drug targets it is often desirable to know which genes, when overexpressed, confer an increased susceptibility to the drug of interest. These genes, which enhance the effect of traditional antibiotics and are not visible in traditional plating strategies, are valuable targets for the design of novel anti-microbial strategies. Second, current micro-array based screening techniques evaluate the effect of a deleted gene upon overall cell physiology (see reference 13). Herein we evaluate the effect of gene overexpression on cell physiology. Similar to the molecular barcoding approach of Shoemaker et al. (see reference 13), however, entire genome libraries are screened in a single heterogeneous growth culture. Finally, most screening technologies rely upon the examination of static time points in selective conditions (see reference 12). Using our technique it is possible to monitor changes in individual plasmid populations throughout growth in various conditions (Dynamic Screening).

Three key criteria were required to provide the evidence necessary for a convincing demonstration of the MADSA; 1) Selective and non-selective conditions had to be identified and the addition of the plasmid library to wild-type cells had to provide a growth advantage and or disadvantage, 2) Plasmid DNA containing E. coli inserts needed to be labeled and hybridized such that the DNA could be effectively identified on the micro-array, and 3) the identified DNA had to reflect the status of the clone population in the selected conditions rather then random hybridization or non-specific hybridization of the plasmid backbone (as demonstrated by reproducibility within groups, differences between groups, and sensibility among identified resistance and susceptibility genes).

(i) Determining Conditions for Pine-Sol Selectivity

E. coli DH5α and DH5α [pTAGL], transformed with an E. coli genomic library (avg. insert 1-2 kbp) prepared from strain MG1655, were grown in Luria Broth (LB) media containing various levels (0-2.5% (v/v)) of the anti-microbial agent Pine-Sol (50 μg/ml of ampicillin was included in DH5α[pTAGL] cultures). Pine-Sol was chosen because it is known to select for mutants also resistant to more traditional antibiotics such as tetracycline, chloramphenicol, and nalidixic acid (see reference 14) and the effects of the indiscriminant use of antimicrobial agents is an issue of continued concern.

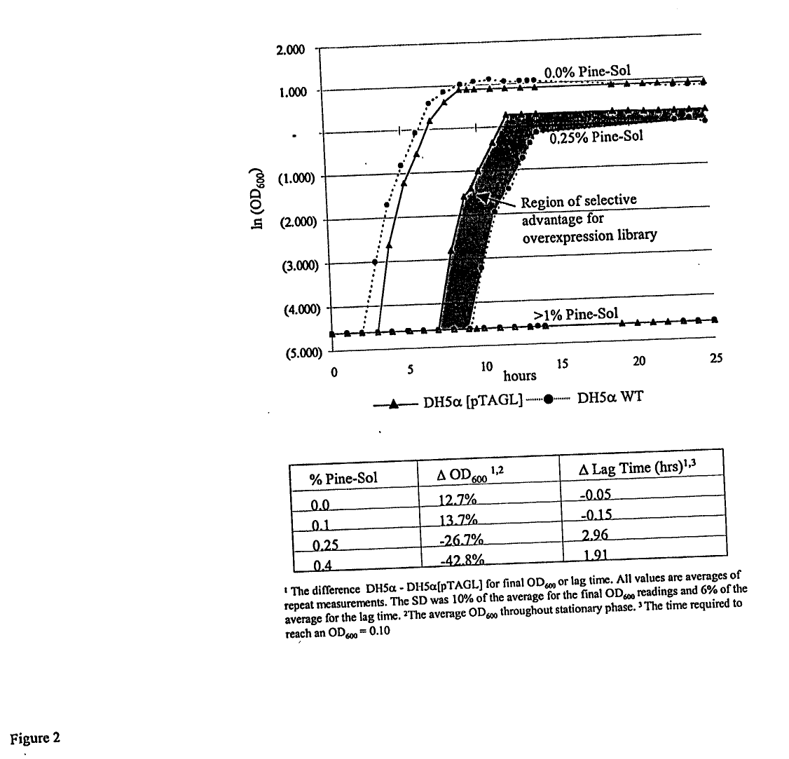

In FIG. 2 growth curves of both DH5α and DH5α[pTAGL] are displayed. At Pine-Sol concentrations less than 0.1% no observable effect on DH5α growth rate was observed. Increasing Pine-Sol concentrations, however, did retard the growth of wild type DH5α as reflected in a decreased final cell density as well as an increased lag time in the wild type cells. At Pine-Sol levels greater 1% (v/v) no growth was observed after greater than 35 hours. At 0.1% (v/v) Pine-Sol, the transformed cells DH5α[pTAGL] did not show a significant difference in final cell density (13%), lag time, or growth rate from the wild type. At 0.25% (v/v) and 0.4% (v/v) Pine-Sol, DH5α[pTAGL] had a decreased lag-time (2-3 hours) and repeatedly grew to higher final densities (27-43%). To understand what members of the genomic library were responsible for the increased Pine-Sol resistance phenotype, we isolated plasmids at various time points from cells grown at 0.0%, 0.1%, 0.25%, and 0.4% (v/v) Pine-Sol in LBA and applied them to the micro-array.

(ii) Probing the Population of a Plasmid Library

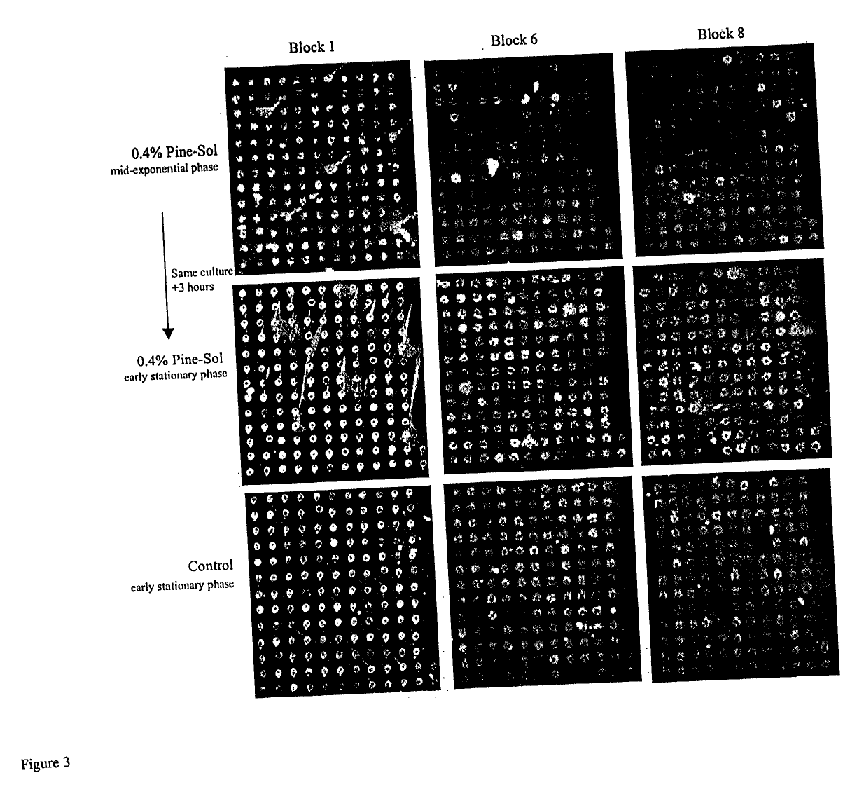

In FIG. 3, three sub-sections of three separate arrays are displayed. The arrays include two time points (mid-exponential and early stationary) within the same 0.4% (v/v) Pine-Sol culture (the most stringent) and the corresponding time point (early stationary) from cells grown without any Pine-Sol. The sub-arrays show approximately 450 of the 1160 genes contained within the arrays.

There are two main features to be obtained from the micro-array images, repeatability within phenotypically distinct groups and reproducible differences between phenotypically distinct groups. By observing reproducible differences, we can eliminate the possibility of non-specific hybridization (should be the same in all groups) and conclude that the array results reflect changing plasmid levels as a function of the cultures they were grown in.

In FIG. 3, several bright semi-smeared spots are visible in the 0.4% samples but not so in the 0.0% samples. These spots provide a quantitative assessment of gene-specific plasmid levels at different time points from the two conditions of interest. They represent plasmids that contained genes selected for in the presence of Pine-Sol but not selected for in identical growth conditions in the absence of Pine-Sol. Arrays of the additional time points from both the 0.4% (v/v) culture (two additional arrays) and the 0.0% culture (two additional arrays) show patterns consistent with those displayed in FIG. 3. To more closely examine the reproducibility of these differences, clustering and principal component analysis was performed (see reference 15). The results revealed three distinct clusters containing a) all four 0.4% samples, b) all three 0% samples, and c) two intermediate (0.1%, 0.25%) samples. Furthermore, in the portions of the sub-arrays not shown in FIG. 3, similar patterns of distinction between selected and non-selected plasmid populations are reproducibly observed (all of these array images can be obtained from the authors upon request, reference web page).

These results clearly demonstrate the enrichment for a subset of the plasmid pool resulting in a non-evenly distributed population of plasmid clones. In fact, the fractional population of the selected plasmids was so high that streaking of the Cy3 signal occurred at the corresponding gene locations on the array. The brightest spots occurred repeatedly in the 0.4% samples on arrays run on separate days and did not occur in any of the 0.0% samples (run at the same time as the 0.4% samples). Therefore, these observations were not the result of differences in hybridization conditions or other systematic errors. Cells grown without any Pine-Sol did not result in such a major change in the distribution of individual plasmids. This is also in agreement with the convention that library amplification can be performed in non-selective conditions and still maintain a relatively stable population of clones. These overarching results demonstrate 1) the reproducibility of micro-array results from samples obtained at different time points within the same culture and 2) the ability of these micro-array results to discriminate between plasmid populations obtained from differentially selected cultures of a whole genome plasmid library.

(iii) Discovering Antibiotic Resistance and Susceptibility Genes

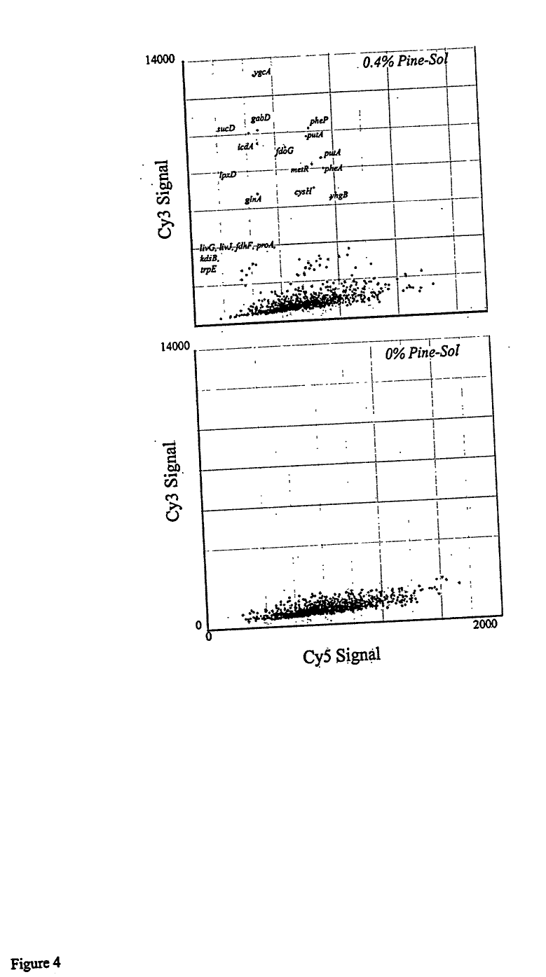

To further understand the biology involved with Pine-Sol resistance and susceptibility we quantified the results from micro-array experiments (14 total arrays). In FIG. 4, the median Cy3 intensity for each gene is displayed versus the median Cy5 intensity for the 0.4% (early stationary) and the 0.0% (early stationary phase) samples shown in FIG. 3. On the same scale, the 0.4% sample contains a more unevenly distributed set of Cy3 signals when compared to the 0.0% samples while the Cy5 signals from both samples are similar. The genes which are responsible for the skewing of this population correspond exactly to the brightest green spots from FIG. 3. The most direct interpretation of these data is that the products of these genes provided a relative growth advantage in the presence of Pine-Sol but did not provide the same relative advantage in the absence of Pine-Sol. Specifically, these genes are Pine-Sol resistance genes.

In FIG. 5, the table of resistance genes reveals a surprising degree of biochemical consistency. Of the 19 identified genes, two (lpxD, kdsB) are involved in lipid A biosynthesis which is known to play a major role in antibiotic resistance and susceptibility (see reference 16, 17). Three of the genes are involved in the transport of branched chain or aromatic amino acids and six are dehydrogenases. An additional two genes have unknown function and with the remaining six of various functionality. Furthermore, eight of the genes have been previously identified as conferring some form of resistance when wild type expression levels are (putA, metR, pheA, cysH, livG, livJ, trpE, proA; (see reference 18)).

The major component of Pine-Sol (Pine-Oil) is α-terpineol (65%) and most of the active components of Pine-Oil are uncharged, non-aromatic cyclic hydrocarbons (see reference 14). The implication from our results is that the mechanism of Pine-Sol action involves amino-acid metabolism and transport. Given the structural similarities of α-terpineol and many metabolites involved in aromatic amino acid biosynthesis and metabolism, this result is sensible. While these implications are not in any way conclusive, they raise the exciting extension of this technique to identify the primary pathways of relevance to the mechanism of action of a particular compound (i.e. toxic compounds, antibiotics, growth enhancing substrates, etc.) (see reference 2).

The presence of six dehydrogenases in the group of identified resistance genes raises some issues of relevance to enzyme evolution. Several groups have revealed evidence that new enzymatic functions can be the result of the transference of an existing cellular chemistry to a new structural backbone (see reference 19). This idea has formed the basis for many of the so-called directed evolution strategies that have resulted in many successful reports of improved enzyme properties (see reference 20). One interpretation of our results is that each of the identified dehydrogenases has a certain degree of activity towards one of the growth retarding by-products of Pine-Sol. The implication is that a bacterium which is able to either increase the level or the specific activity of one of these dehydrogenases towards the antibiotic of interest will succeed in becoming an antibiotic resistant strain. The development of resistance through the cellular application of a pool of supra-family enzymatic chemistries is an intriguing notion worthy of additional investigation.

To identify antibiotic susceptibility genes a more rigorous test of plasmid population differences was required. Specifically, some overexpressed genes lead to a loss of viability regardless of the media in which the cells are grown. Such genes would not be detected in the MADSA but of course do not represent a realistic resistance mechanism. What we were interested in identifying were those genes that conferred a loss of viability when overexpressed in cells grown in the presence of Pine-Sol but that grew normally when Pine-Sol was not added to the media. To do so, we compared the mean intensity value for each gene in four samples taken over 1.5 hr intervals from cells grown in the presence of 0.4% Pine-Sol to three time-course samples from cells grown in the absence of Pine-Sol (0.0% samples). We defined significantly different as those genes whose means had a greater than 95% chance of being lower in the selective media than in the Pine-Sol free media (p<0.05). FIG. 5 contains a list of 24 genes that passed this test at the 95% confidence level.

While many of the identified genes made sense from a antibiotic susceptibility perspective, the presence of the rfaC and rfaD genes was very encouraging. These two genes are part of a class of so called rough genes whose altered function is well known to be associated with hyper-susceptibility (see reference 21). An additional six genes have products involved in membrane based transport activities (oppF, ptsI, ptsN, malX, cydA, cysU) (see reference 16). Furthermore, nine of the genes (atpG, metK, upp, ptsI, codA, nadB, serA, cydA, pFkA) are known as selectable markers when their levels or structures are altered compared to wild type (see reference 18). Considering the presence of three genes of unknown function, more than half of the identified susceptibility genes are justified based on known behavior or function. A final note concerns the well known susceptibility genes ompF and ompC. These two genes encode for outer membrane porins whose altered function produces hyper-susceptibility (see reference 22). For both of these genes, the average intensity value in the 0.4% Pine-Sol samples was considerably lower than in the un-selected population. For OmpC and OmpF confidence could be assigned at approximately 85% (p=0.146) and 70% (p=0.316), respectively. While these levels did not confer to the relatively stringent 95% confidence interval, plasmids containing the genes for these two porins did show a reduced growth rate in the presence of Pine-Sol when compared the absence of Pine-Sol.

V. References

1. C. Roberts, et al., Science 287, 873-880 (2000).

2. M. Marton, et al., Nature Medicine 4, 1293-1301 (1998).

3. L. Gambino, S. Gracheck, P. Miller, Journal of Bacteriology 175, 2888-94 (1993).

4. C. Khosla, J. Bailey, Nature 331, 633-634 (1988).

5. C. Henry, Chemical and Engineering News, 41-58 (2000).

6. D. Heymann, J. Koplan, “Overcoming Antimicrobial Resistance” (World Health Organization, 2000).

7. www.tigr.org, (Obtained from The Institute for Genomics Research.).

8. E. coli genomic DNA was prepared using Qiagen Genomic Tips (Valencia, Calif.). Purified DNA was fragmented using sonication for 30 seconds. Fragmented DNA was size separated through a 1% agarose gel and fragments between 0.5-3.5 kbp were extracted (Qiagen). Extracted DNA was repaired using T4 DNA polymerase and Klenow fragment then dephosphorlyated using Calf-Intestinal Phosphatase. dATP was added to the 3′ ends of repaired DNA using Taq DNA polymerase and ligation was performed using the TOPO-TA pBAD cloning vector from Invitrogen. Ligation products were electroporated into electroporation competent DH5α cells (Invitrogen) and plated on LB +Ampicillin (LBA). Transfornants were grown overnight at 37 C. and harvested by adding LBA directly to the plates and amplifying for 3.5 hours at 37 C. in 300 ml of LBA at 250 rpm. Plasmids were harvested using a Qiagen Maxi Kit.

9. At an average insert size of 1.6 kbp 12,000 colonies corresponds to a 99% probability that all sequences were included in the original library.

10. Plasmids were purified by Qiagen Mini Prep Kits (Valencia, Calif.). Purified plasmids were sonicated for 35 seconds using a 1 second pulse cycle. Fragmented plasmids were checked by 1% agarose gel to confirm a size distribution of 0.5-1.5 kbp relative to an unfragmented plasmid size distribution of 4.5-7 kbp. Fragmented plasmids were labeled using the general random primed labeling methodology. 2 ug of plasmid DNA was mixed with 25 nM each of dATP, dCTP, dGTP, 10 nM dTTP, 40 nM Cy3-dUTP, 50 U of Klenow Enzyme (3′-5′), and Klenow Buffer and incubated for 1 hour at 37 C. Labeled plasmids mixed with an equal amount of identically and concurrently Cy5 labeled genomic DNA.

11. The labeled plasmid and genomic DNA mixture was purified from unincorporated nucleotides through a DyeEx spin column (Qiagen), and ethanol precipitated at −20 C. for 1 hour. Labeled pellets were resuspended in 18 ul of hybridization solution (Clontech), denatured for 10 minutes at 95 C., and applied to the arrays.

12. R. Cho, et al., Proceedings of the National Academy of Sciences 95, 3752-3757 (1998).

13. D. Shoemaker, D. Lashkari, D. Morris, M. Mittmann, R. Davis, Nature Genetics 14, 450-456 (1996).

14. M. Moken, L. McMurry, S. Levy, Antimicrobial Agents and Chemotherapy 41, 2770-72 (1997).

15. Clustering and Principal Component Analysis (PCA) were performed using MATLAB. In the clustering analysis, the similarity was set to the correlation coefficient and the average linkage technique was used. Clustering results were compared to PCA results to determine the number of clusters contained within the samples. After it was determined that 2-3 clusters were apparent, PCA was used to eliminate samples that did contribute much to the overall variance of the micro-array data (eliminated five samples with the lowest loadings in both PC1 and PC2). The remaining 9 samples were then re-run through the clustering and PCA to reveal two general classes corresponding to the 0.4% Pine-Sol treated samples (class 1) and the samples treated with 0.25% (1 sample), 0.1% (1 sample), or 0.0% Pine-Sol (3 samples) (class 2). Within class 2, those cells treated with Pine-Sol split from the 0.0% samples to form a distinct cluster. A similar result was obtained in the PCA where PC2 values greater than zero contained only samples from 0.4% Pine-Sol.

16. M. Berlyn, in Escherichia coli and Salmonella: Cellular and Molecular Biology 2nd Edition F. C. Neidhardt, Ed. (American Society for Microbiology, Washington D.C., 1996) pp. 1753-1805.

17. M. Vaara, M. Nurminen, Antimicrobial Agents and Chemotherapy 43, 1459-1462 (1999).

18. R. LaRossa, in Escherichia coli and Salmonella: Cellular and Molecular Biology 2nd edition F. Neidhardt, Ed. (American Society for Microbiology, Washington D.C., 1996) pp. 2527-2587.

19. P. Babbitt, et al., Science 267, 1159-61 (1995).

20. A. Crameri, S. A. Raillard, E. Bermudez, W. Stemmer, Nature 391, 288-290 (1998).

21. M. Vaara, Antimicrobial Agents and Chemotherapy 37, 2255-60 (1993).

22. H. Nikaido, in Escherichia coli and Salmonella: Cellular and Molecular Biology 2nd Edition F. C. Neidhardt, Ed. (American Society for Microbiology, Washington D.C., 1996) pp. 29-47.

23. M. Schena, Shalon, D., Davis, R., and Brown, P., Science 270, 467-470 (1995).

24. J. DeRisi, Iyer, V., Brown, P., Science 278, 680-685 (1997).

We claim:

1. A method for identifying one or more genetic elements which selectively confer a target phenotype on a target cell, comprising:
(i) generating a library of expression vectors comprising a variegated population of genetic elements from a population of genomic fragments;
(ii) transfecting host cells with said library of expression vectors;
(iii) subjecting the host cells to selective growth conditions under which genetic elements are expressed;
(iv) isolating and/or amplifying a sub-population of host cells based on having the target phenotype during selective growth conditions;
(v) differentially labeling and admixing genetic elements from said sub-population of host cells with genomic fragments and contacting the admixture with one or more oligonucleotide arrays; and
(vi) identifying coding sequences which confer said phenotype by hybridization of said genomic sequences to said one or more oligonucleotide arrays.

2. A method for identifying one or more genetic elements which selectively confer a target phenotype on a target cell, comprising:
(i) transfecting host cells with a library of expression vectors comprising a variegated population of coding sequences for genetic elements;
(ii) subjecting the host cells to selective growth conditions under which said genetic elements are expressed;
(iii) isolating and/or amplifying a sub-population of host cells based on having the target phenotype during selective growth conditions;
(iv) contacting coding sequences for said genetic elements from said sub-population of host cells with one or more oligonucleotide arrays; and
(v) identifying individual coding sequences which confer said phenotype by hybridization to said one or more oligonucleotide arrays.

3. The method of claim 2, wherein the variegated population of coding sequences for genetic elements includes a genomic fragment library.

4. The method of claim 2, wherein the variegated population of coding sequences for genetic elements includes a cDNA library.

5. The method of claim 2, wherein the host cell is a prokaryotic cell.

6. The method of claim 2, wherein the host cell is a eukaryotic cell.

7. The method of claim 6, wherein the host cell is a mammalian cell.

8. The method of claim 2, wherein the expression vector is an episomal vector.

9. The method of claim 2, wherein the expression vector is an intregrative vector.

10. The method of claim 2, wherein said target phenotype is a change in expression of a reporter gene.

11. The method of claim 2, wherein said target phenotype is a change in expression of a marker protein.

12. The method of claim 2, wherein said target phenotype is ability to growth under the selective conditions.

13. The method of claim 2, wherein said target phenotype is change in cell morphology.

14. The method of claim 2, wherein said sub-population of host cells is isolated by flow cytometry based on expression of a reporter gene or marker protein.

15. The method of claim 2, wherein said genetic elements encode polypeptides.

16. The method of claim 2, wherein said genetic elements encode antisense RNA.

17. A method of conducting a drug discovery business comprising:
(i) identifying, by the assay of claim 2, a target gene which confers said target phenotype;
(ii) identifying agents by their ability to inhibit expression of the target gene or the activity of an expression product of the target gene in order to inhibit said target phenotype in a target cell or tissue;
(iii) conducting therapeutic profiling of agents identified in step (b), or further analogs thereof, for efficacy and toxicity in animals; and
(iv) formulating a pharmaceutical preparation including one or more agents identified in step (iii) as having an acceptable therapeutic profile.

18. The method of claim 17, including an additional step of establishing a distribution system for distributing the pharmaceutical preparation for sale, and may optionally include establishing a sales group for marketing the pharmaceutical preparation.

19. A method of conducting a target discovery business comprising:
(i) identifying, by the assay of claim 2, one or more target genes which confer said target phenotype;
(ii) (optionally) conducting therapeutic profiling of the loss-of-function phenotypes of said target gene for potential efficacy and toxicity in animals; and
(iii) licensing, to a third party, the rights for further drug development of inhibitors of said target gene(s).
